(19) United States
(12) Patent Application Publication
(10) Pub. No.: US 2017/0247751 A1
(43) Pub. Date: Aug. 31, 2017

(54) APPARATUS AND METHODS FOR DETECTING MULTIPLE LABELLED BIOPOLYMERS

(71) Applicant: Microscale Devices LLC, Morrisville, NC (US)

(72) Inventor: John A. Humphrey, Morrisville, NC (US)

(21) Appl. No.: 15/500,452

(22) PCT Filed: Jul. 27, 2015
(86) PCT No.: PCT/US15/42164

§ 371 Date: Jan. 30, 2017

Related U.S. Application Data
(60) Provisional application No. 62/029,093, filed on Jul. 25, 2014.

(51) Int. Cl. C12Q 1/68; G01N 21/64; B01L 3/00

(52) U.S. Cl. CPC C12Q 1/6827; B01L 3/50851; C12Q 1/6825; G01N 21/6428; G01N 21/645; B01L 2300/0654; B01L 2300/0681; G01N 2021/6421; G01N 2021/6439; G01N 2021/6471

(57) ABSTRACT

The disclosure is directed toward instrumentation/processes for generating multiple datapoints from a sample of multiple comingled labeled biopolymers. In one embodiment, it relates the description of optical approaches used to integrate the samples, the optical approaches used to alter the light path along the “Z” axis perpendicular to the “X” and “Y” axes of a plate containing multiple samples in multiple sample wells and thereby provide greater functionality and the integration of these approaches with purification methods. Alternatively, it provides methods of analyzing beads with multiple labeled nucleic acids attached.

### CROSS REFERENCE TO RELATED APPLICATIONS

This application claims the benefit of Ser. No. 62/029,093 filed Jul. 25, 2014, John Humphrey, Atty. Dkt. 448/2 PROV which is hereby incorporated by reference in its entirety.

### FIELD

This disclosure relates to an apparatus and/or process for assaying multiple comingled fluorescently labeled biopolymers, e.g., nucleic acid species. In one embodiment, it may be used to create multiple datapoints from a single DNA sample.

### BACKGROUND

### Introduction

There are many modem molecular biology techniques devoted to generating multiple datapoints from a single nucleic acid sample. As a general principle, these techniques either involve segregating and amplifying individual pieces of DNA to create unique addressable amplicons [e.g. genotyping by sequencing (Andolfatto et al. “Multiplexed shotgun genotyping for rapid and efficient genetic mapping” 2011, Genome Res 21′. 610-17; Baird et al. “Rapid SNP discovery and genetic mapping using sequenced RAD markers” 2008, PLoS One 3: e3376; Elshire et al. “A Robust, Simple Genotyping-by-Sequencing (GBS) Approach for High Diversity Species” 2011 PLoS One Q- e19379)] or mass amplification of multiple different amplicons followed by physical separation of the ampicons via the unique physical properties of the individual amplicons (e.g. mass spec (U.S. Pat. No. 5,605,798 A), hybridization sequence (U.S. Pat. No. 6,045,996)).

Fluorescence assays that use multiple probes typically require measuring the fluorescence emission levels in two spectral bands, corresponding to the probes involved. Since there are no known fluorophores which have a single emission wavelength, using traditional optical configurations severely limits the number of different fluorophores which may be imaged simultaneously in one physical space. There are however a variety of nontraditional techniques by which multiple fluorophores may be imaged simultaneously in the same physical space. For example fixed filters, a filter wheel, a diffraction grating (U.S. Pat. No. 3,237,508) or tunable filters such as acousto-optic tunable filters (“AOTF”) (U.S. Pat. No. 3,749,476) or liquid crystal tunable filters (“LCTF”) (U.S. Pat. No. 6,403,947, U.S. Pat. No. 6,455,861 B1, US 2002/0070349 A1) may all be used to temporally and sequentially limit the emission bands which reach the detector. This combination of temporal, sequential limitation of spectral bands to a specific geography creates a spectral signature which enables visually similar fluorophores to be differentiated on the basis of their optical properties across the assayed spectrum rather than their most intense spectral feature.

### SUMMARY

In particular non-limiting embodiments, the present disclosure provides for a manner to quantitate and identify multiple molecular markers in the same physical geography. This disclosure provides a system for analyzing multiple labeled nucleic acids in a sample, each labeled nucleic acids being labeled by a covalent linkage with at least one synthetic fluorescent molecule capable of generating emitted light at one or more light frequencies in response to an exciting light, the system comprising: a light source configured to produce an exciting light; an excitation filter; a dichroic mirror capable of directing the exciting light to the sample; a tunable emitted light filter capable of filtering the plurality of emitted light frequencies from the sample; and a detector capable of detecting the plurality of emitted light frequencies from the multiple labeled nucleic acids in the sample wherein the tunable emitted light filter is at about a 0 to 45 degree angle or a 135 to 180 degree angle to the directed exciting light and the sample.

The system of paragraph 0005, wherein the tunable emitted light filter is about a 5 to 25 degree angle, a 10 to 20 degree angle, a 155 to 175 degree angle, a 160 to 170 degree angle to the directed exciting light and the sample. Alternatively, in embodiments where the emitted light is collected on the opposite side of the sample from the directed light, e.g., parallel or approaching to the detector. Here, the tunable emitted light filter is about a 0 to 5 degree angle, a 5 to 10 degree angle, a 10 to 15 degree angle, a 15 to 20 degree angle, a 25 to 25 degree angle, a 25 to 30 degree angle, a 30 to 35 degree angle, a 35 to 40 degree angle, or a 40 to 45 degree angle to the directed exciting light and the sample. In other embodiments, the emitted light may be collected on the same side of the sample as the directed exciting light, e.g, the excitation and emission are from above. In this embodiment, the tunable emitted light filter is about a 175 to 180 degree angle, a 170 to 175 degree angle, a 165 to 170 degree angle, a 160 to 165 degree angle, a 165 to 160 degree angle, a 160 to 155 degree angle, a 155 to 150 degree angle, a 150 to 145 degree angle, a 145 to 140 degree angle, a 140 to 135 degree angle, to the directed exciting light and the sample. I

The system of paragraph 0005 or 0006, wherein the tunable emitted light filter is a solid state tunable light filter such as a liquid crystal tunable light filter.

The system of paragraph 0005, 0006, or 0007 wherein each labeled nucleic acid further comprises a purification tag such as a synthetic polypeptide designed to bind a specific antibody or a metal binding polypeptide.

The system of paragraph 0005, 0006, or 0007, wherein the light source is a plurality of lasers.

A system for analyzing multiple labeled nucleic acids in a sample, the labeled nucleic acids being capable of generating emitted light at a plurality of light frequencies in response to an exciting light, the system comprising: a light source configured to produce an exciting light; an excitation filter; a multi-part prism capable of directing the exciting light to the sample; a tunable emitted light filter capable of filtering the plurality of emitted light frequencies from the sample; and a detector capable of detecting the plurality of emitted light frequencies from the multiple labeled nucleic acids in the sample.

A method of analyzing multiple labeled nucleic acids in a sample, the labeled nucleic acids being capable of generating a plurality of emitted light frequencies in response to an exciting light, the method comprising: producing an exciting light from a light source; directing the light source through an excitation filter; directing the filtered excitation light through a dichroic mirror capable of directing the exciting light to the sample; generating the plurality of emitted light frequencies from the sample and directing the emitted light to a tunable emitted light filter; directing the filtered emitted light to a detector capable of detecting plurality of emitted light frequencies from the multiple labeled nucleic acids in the sample.

The apparatus or method of any of paragraphs 0005-0011, wherein the multiple labeled nucleic acids are multiple labeled DNA molecules.

The apparatus or method of any of paragraphs 0005-0012, wherein the multiple labeled DNA molecules are amplicons from a PCR reaction.

The apparatus or method of any of paragraphs 0005-0013, wherein the multiple labeled nucleic acids are designed to be specific for a single nucleotide polymorphism (SNP).

The apparatus or method of any of paragraphs 0005-0014, wherein the sample is a bead or other solid substrate.

The apparatus or method of any of paragraphs 0005-0015, wherein the bead has a plurality of nucleic acids non-covalently bound to the bead or other solid substrate

The apparatus or method of any of paragraphs 0005-0014, wherein the sample is a homogenous solution in a multi-welled plate or other collection/storage device.

### BRIEF DESCRIPTION OF THE FIGURES

FIG. 1—describes the use of a tunable filter in a process in which both excitation and emission of the fluorescently labeled DNA occurs from above.

FIG. 2—describes the use a tunable filter in a process detecting fluorescently labeled DNA in which the light source is parallel to the detector.

FIG. 3—describes the use of a tunable filter in a process detecting fluorescently labeled DNA in which the light source is perpendicular to the detector.

FIG. 4—describes a bead based purification scheme for the PCR products prior to multi spectral imaging (MSI).

FIG. 5—describes a multiplex purification scheme prior to multi spectral imaging.

### DETAILED DESCRIPTION

The current disclosure pertains to a process by which multiple different amplicons present in the same spatial geography and/or on the same solid substrate (e.g., bead, slide, fiber, plate) may be imaged simultaneously using their fluorescent properties.

PCR has become one of the most widely used techniques in molecular biology. The purpose of this process is to create short copies of DNA called amplicons from original template DNA. This process is iterative—strands of DNA are copied and then these copies, along with the original DNA, are used to generate other copies in subsequent cycles. Under ideal conditions this process is geometric. There are many elements required for a successful PCR reaction: “seed” or template DNA, primers, dNTPs, salts (especially magnesium), and polymerase are all part of the minimum requirements for a PCR reaction. In addition to the chemical requirements PCR reactions require specific vessels designed to hold liquid and provide heat transfer of heat between the liquid in the vessel and the surrounding environment aka PCR block, water bath or air (U.S. Pat. No. 4,683,195A) (Saiki et al., Science 239:487, 1988).

The combination of tunable filters with PCR provides the ability to create an apparatus for fluorescence detection of two or more differentially fluorescent amplicons/DNA at a single spatial geography. This apparatus will be associated with a process composed of at least two steps: the first step will consist of a labeling step in which the DNA will be fluorescently labeled, the second step will consist of a multi spectral imaging step (MSI). It is important to note that the multi spectral imaging step will not occur on a PCR reaction (if one is used in the labeling process) rather it will only occur on a pool of fluorescently labeled DNA. Finally the MSI process will only be applied to the light emitted from the pool of DNA not the light used to excite the DNA.

Apparatus/process for assaying multiple comingled fluorescently labeled nucleic acid species outside of a PCR reaction with or without a PCR reaction proceeding the analysis. An apparatus/process for fluorescence detection of two or more differentially labeled fluorescent DNA amplicons at a single spatial geography.

An apparatus which contains a manner of altering the geography which is being integrated by altering the path of the excitation light. An apparatus which contains a means of altering the integrated geography by using a dichroic mirror to direct the excitation light almost straight down and allowing the emitted light to pass to a detector at approximately a right angle to the light source. An apparatus in which a detector is at a right angle to a vessel/solid support and simultaneously at a right angle to the light source and the advantages that this configuration provides. A method of purification which enables PCR products to be separated from the fluorescently labeled oligos used to create the amplicons and the advantages that this provides.

A method using multiple different types or levels of purification enabling multiple different PCR products to be purified from the same PCR reaction. A method of combining these purification processes with an altered pathway for the beam of light.

### Definitions

In one embodiment, the light source is a plurality of lasers. The lasers may be diode-pumped solid-state (DPSS) lasers including but not limited to frequencies of 320, 355, 435, 457, 473, 480, 491, 500.8, 514.5, 515, 523.5, 526.5, 532, 543, 556, 561, 589, 593.5, 604, 607, 656.5, 660, 671, 722, 914, 946, 1030, 1047, 1053, 1064, 1085, 1112, 1122, 1313, 1319, 1342, or 1444. Alternatively, the laser may be a single-frequency, single longitudinal mode diode-pumped solid-state, Q switched or a lamp pumped solid state diode pumped lasers including but not limited to frequencies 457, 473, 532, 561, 589, 671, 1064, 1342, 266, 355, 523.5, 526.5, 532, 656.5, 660, 1047, 1053, 1064, 1313, 1319, 1573, 3800. It may be a collimated diode laser including but not limited to wavelengths (375, 405, 442, 447, 450, 454, 462, 488, 520, 520, 633, 635, 637, 640, 658, 680, 685, 690, 705, 730, 785, 793, 800, 808, 825, 830, 845, 852, 880, 885, 915, 940, 965, 975, 980, 1060, 1450, 1470, 1550, 1870, 1908, 1940, 2200). The laser may be a gas laser including but not limited to helium/neon, argon, krypton, xenon, nitrogen, carbon dioxide, carbon monoxide and eximer lasers, dye lasers including but not limited to stilbene, courmanin, rhodamine lasers, chemical lasers including but not limited to hydrogen fluoride, deuterium fluoride, chemical oxygen/iodine, all gas iodine lasers metal lasers including but limited to helium-cadmium, helium-mercury, helium-selenium, helium-silver, strontium vapor, neon, neon copper, copper vapor, gold vapor and solid state lasers including but not limited to ruby, titanium—sapphire, cesium lithium strontium or calcium aluminum fluoride laser, alexandrite lasers.

Other light sources including but not limited to flash lamps including but not limited to xenon flash lamps, mercury vapor lamps with and without emission filters and LED light sources with and without emission filters.

Nucleic acid molecules suitable for use in the methods and apparatus of this disclosure include DNA and RNA in both single-stranded and double-stranded form, as well as the corresponding complementary sequences. An “isolated nucleic acid” is a nucleic acid that has been separated from adjacent genetic sequences present in the genome of the organism from which the nucleic acid was isolated, in the case of nucleic acids isolated from naturally-occurring sources, in the case of nucleic acids synthesized chemically, such as oligonucleotides, or enzymatically from a template, such as polymerase chain reaction (PCR) products or cDNAs, it is understood that the nucleic acids resulting from such processes are isolated nucleic acids. An isolated nucleic acid molecule refers to a nucleic acid molecule in the form of a separate fragment or as a component of a larger nucleic acid construct. A sample comprising nucleic acid in a form suitable for hybridization with a probe, such as a sample comprising nuclei or nucleic acids isolated or purified from such nuclei. The nucleic acid sample may comprise total or partial (e.g., particular chromosome(s)) genomic DNA, total or partial mRNA (e.g., particular chromosome(s) or gene(s)), or selected sequence(s).

“Probe,” in the context of the present disclosure, is an oligonucleotide or polynucleotide that can selectively hybridize to at least a portion of a target sequence under conditions that allow for or promote selective hybridization. In general, a probe can be complementary to the coding or sense (+) strand of DNA or complementary to the non-coding or anti-sense (−) strand of DNA (sometimes referred to as “reverse-complementary”). Probes can vary significantly in length. A length of about 10 to about 100 nucleotides, such as about 15 to about 75 nucleotides, e.g., about 15 to about 50 nucleotides, can be preferred in some applications such as PCR, whereas a length of about 50 to about 1×10⁶ nucleotides can be preferred for chromosomal probes and a length of about 5,000 to about 800,000 nucleotides or more preferably about 100,000 to about 400,000 for BAC probes.

Single nucleotide polymorphism (SNP) is a DNA sequence variation occurring either within the population at large or between paired chromosomes. In addition to the strict definition of this term as a single nucleotide of difference, for the sake of ease of reading throughout this document, SNP can also refer to other molecular markers commonly used in genetic mapping such as InDels and microsatellites. Finally SNP can also refer to techniques to detect methylation patterns; specifically those detectable by methylation specific PCR. (Herman, J G; et al, (1996) PNAS 93:9821-9826)

Microsatellite—also known as simple tandem repeats, short tandem repeats, variable tandem repeats. With variations allowed for length of the repeat, the number of repeated units and the inter repeat consistency, microsatellites consist of “short” nucleotide sequences that are repeated contiguously.

PCR is a process in which a small piece of “seed” DNA is amplified into much larger numbers of “amplicons”. These amplicons are created in the presence of: “seed” or template DNA, primers of sufficient proximity, concentration and orientation, dNTPs, salts (especially magnesium), and polymerase subjected to the sequential application of heat for predetermined intervals of time.

Fluorescence refers to the process by which radiation—typically light—is applied to a material at certain wavelength(s) and then radiation of different wavelengths—typically lower wavelengths and typically light—is emitted by that material.

InDels refer to insertions/deletions. These are specific interpopulation or intrapopulation genetic features in which one nucleotide to kilobases of nucleotides are present in one individual that are not typically found in the population or are found one but not both paired chromosomes.

Functional group—throughout this document purification schemes are presented via functional groups such as biotin. It is important to note that term functional groups can also refer to other selection properties such as size, charge, hydrophobicity/hydrophilicity that can be used for selection.

### Polynucleotide Sequence Amplification and Determination

In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits of the disclosure may use any nucleic acid amplification or detection methods known to one skilled in the art, such as those described in U.S. Pat. Nos. 5,525,462 (Takarada et al.); 6,114,117 (Hepp et al.); 6,127,120 (Graham et al.); 6,344,317 (Urnovitz); 6,448,001 (Oku); 6,528,632 (Catanzariti et al.); and PCT Pub. No. WO 2005/111209 (Nakajima et al.); all of which are incorporated herein by reference in their entirety. Commonly used methods known in the art for the quantification of mRNA expression in a sample include northern blotting and in situ hybridization (Parker and Barnes, Methods Mol. Biol. 106:247-83, 1999), RNAse protection assays (Hod, Biotechniques 13:852-54, 1992), PCR-based methods, such as reverse transcription PCR (RT-PCR) (Weis et al., TIG 8:263-64, 1992), and array-based methods (Schena et al., Science 270:467-70, 1995). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE), bead-based technologies, single molecule fluorescence in situ hybridization (smFISH) studies, and gene expression analysis by massively parallel signature sequencing. Velculescu et al. 1995 Science 270 484-487; Streefkerk et al., 1976, Pro Biol Fluid Proc Coll 24 811-814; Soini U.S. Pat. No. 5,028,545; smFISH, Lyubimova et al. 2013 Nat Protocol 8(9) 1743-1758.

In some embodiments, the nucleic acids are amplified by PCR amplification using methodologies known to one skilled in the art. One skilled in the art will recognize, however, that amplification can be accomplished by any known method, such as ligase chain reaction (LCR), QP-replicase amplification, rolling circle amplification, transcription amplification, self-sustained sequence replication, nucleic acid sequence-based amplification (NASBA), each of which provides sufficient amplification. Branched-DNA technology may also be used to qualitatively demonstrate the presence of a sequence of the technology, which represents a particular methylation pattern, or to quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; U.S. Pat. No. 4,683,202 (Mullis); which are incorporated herein by reference in their entirety. PCR reagents and protocols are also available from commercial vendors, such as Roche Molecular Systems. PCR may be carried out as an automated process with a thermostable enzyme. In this process, the temperature of the reaction mixture is cycled through a denaturing region, a primer annealing region, and an extension reaction region automatically. Machines specifically adapted for this purpose are commercially available.

### Samples

The sample may be derived from any and all components of a human including but not limited to blood, hair, feces, tissues, saliva; any and all components of an animal including but not limited to blood, hair, feces, tissues, saliva, feathers, hooves, fins, combs; and any and all components of a plant including but not limited to leaves, roots, stems, seeds, flowers, fruits, vegetables, tubers from agricultural and non-agricultural plants cultivated or uncultivated. In addition a bioproduction vessel and its contents, cultured cells, environmental samples including but not limited to soil samples, water samples and air samples; sewage samples, microbiome and microbiota samples from humans, soil, plants and animals are all covered. All analytes of the sample DNA, RNA and protein are included. In addition means of assaying the phosphorylation or glycosylation status of the protein is specifically covered by this application.

This disclosure provides compositions and kits for detecting genetic polymorphisms including SNPs, InDels, microsatellites and other genetic features. This disclosure also includes a variety of different purification methods including but not limited to biotin/streptavidin and similar pairs; antibodies/antigens and similar pairs; lectins/glycoforms and similar pairs; chemical entities like small molecules/proteins and similar pairs; photosensitive chemicals and their binding partners; defined protein purification elements like histidine tags, thiophilic resins and their binding elements.

A labeled nucleic acid may include one or more regions of nucleotides that do not hybridize to the base nucleic acid. Each labeled nucleic acid may be detected by any convenient detection process capable of detecting an electromagnetic signal generated by each label (e.g., a visible light signal, a UV light signal, or an IR signal). In addition, probes and amplicons may be detected based upon their intrinsic fluorescent/autofluorescent or spectral properties. For example, a charge coupled device (CCD) photodetector or photomultiplier tube (PMT) can be used to detect signals from one or more distinguishable fluorescent probes or quantum dots (QDs).

This disclosure encompasses any method known in the art for amplifying the nucleic acids to enhance the sensitivity of the detectable signal in such assays, including, but not limited to, the use of cyclic probe technology (Bakkaoui et al., 1996, BioTechniques 20: 240-8); and the use of branched probes (Urdea et al., 1993, Clin. Chem. 39, 725-6). The hybridization complexes are detected according to well-known techniques in the art. Reverse transcribed or amplified nucleic acids may be modified nucleic acids. Modified nucleic acids can include nucleotide analogs, and in certain embodiments include a detectable label and/or a capture agent. Examples of detectable labels include, without limitation, fluorophores, radioisotopes, colorimetric agents, light emitting agents, chemiluminescent agents, light scattering agents, enzymes and the like.

The amplification and/or purification may involve a capture agents include, without limitation, an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments.

A signal from the labeled nucleic acid may be analyzed, for example, using a spectrophotometer to detect color from a chromogenic substrate; or a fluorometer to detect fluorescence in the presence of light of a certain wavelength. If desired, the assays of the present disclosure can be automated or performed robotically, and the signal from multiple samples can be detected simultaneously.

Compositions and Kits

This disclosure provides compositions and kits for multiplexed measurement of nucleic acids. Kits for carrying out the assays of this disclosure typically include, in suitable container means, (i) a probe that comprises a nucleic acid sequence that specifically binds to the polynucleotides of interest, (ii) a label for detecting the presence of the probe and (iii) instructions for how to measure the levels. The container means of the kits will generally include at least one vial, test tube, flask, bottle, syringe and/or other container into which a first antibody specific for one of the polypeptides or a first nucleic acid specific for one of the polynucleotides of the present disclosure may be placed and/or suitably aliquoted. Where a second and/or third and/or additional component is provided, the kit will also generally contain a second, third and/or other additional container into which this component may be placed. Alternatively, a container may contain a mixture of more than one antibody or nucleic acid reagent, each reagent specifically binding a different marker in accordance with the present disclosure. The kits of the present disclosure will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present disclosure.

Labeled Nucleic Acids

The labeled nucleic acid may be labels with a luciferase (firefly, bacterial, or coelenterate), green fluorescent protein, a near-infrared fluorophore for near- infra red (near-IR) label, or other luminescent molecule. Such labeled nucleic acids may include a fluorescent moiety, such as a fluorescent protein, peptide, or fluorescent dye molecule. The fluorescent moiety may be a derivative of a naturally-occurring fluor such as nicotinamide co-factors such as NADH; phenylalanine, quinine, or tyrosine. Common classes of synthetic fluorescent molecules useful herein include, but are not limited to, acridinones such as 7-hydroxy-9H-(1,3-dichloro-9,9-dimethylacridin-2-one; aromatic amines such as dansyl; anthracenes; ATTO labels (ATTO-TEC Gmbh Siegen, Germany) such as ATTO 390, ATTO 425, ATTO 465, ATTO 488, ATTO 495, ATTO 520, ATTO 532, ATTO 550, ATTO 565, ATTO 590, ATTO 594, ATTO 610, ATTO 611X, ATTO 620, ATTO 633, ATTO 635, ATTO 637, ATTO 647, ATTO 647N, ATTO 655, ATTO 665, ATTO 680, ATTO 700, ATTO 725, or ATTO 740; benzofurans; bimanes; boron dipyrromethyl phenoxazine (BODIPY) such BODIPY-FL, BODIPY-TR, a resorufin or resorufin N-oxide; butadienes; carbazoles; coronenes; coumarins and their derivatives such as umbelliferone and aminomethyl coumarins such as 4-methyl-7-hydroxy-coumarin, analine derivatives of 7-aminocoumarin; cyanines such as cyanine dyes, e.g., Cy3, Cy3B, Cy3.5, Cy5, Cy5.5, Cy7; dicyanomethylene pyranes; lanthanide metal chelate complexes; oxabenzanthrane, polymethines; pyrylium, perylenes; phenanthrecene, pyrenes; quantum dots (QDs) see Resch-Genger et al. 2008 Nat Meth 5(9) 763-775; quinacridones; rare-earth metal chelate complexes; rubrenes; stilbenes; squarate dyes such as U.S. Pat. Nos. U.S. Pat. Nos. 4,806,488 (Berger et al.); 6,140,494 (Hamilton et al.); xanthenes such as rhodamines, rhodols and fluoresceins, and their derivatives; and derivatives of such dyes. The label may be a fluorescein dye such as a 5-carboxyfluorescein, 6-carboxyfluorescein; and fluorescein-5-isothiocyanate, see U.S. Pat. Nos. 4,439,356 (Khanna and Colvin); 5,066,580 (Lee), 5,750,409 (Hermann et al.); and 6,008,379 (Benson et al.). The label may be a rhodamine dye, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®, tetramethylrhodamine 5-carboxamido-(6-azidohexanyl))(TAMRA), and other rhodamine dyes and U.S. Pat. Nos. 5,936,087 (Benson et al.), 6,025,505 (Lee et al.); 6,080,852 (Lee et al.).

Fluorescent dyes are discussed, for example, in Lavis and Raines 2014 ACS Chem Biol 9 855-866; Lavis and Raines 2008 ACS Chem Biol 3(3) 142-155; Vendrell et al. 2012 Chem Rev 112 4391-4420; U.S. Pat. Nos. 4,452,720 (Harada et al.); 5,227,487 (Haugland and Whitaker); and 5,543,295 (Bronstein et al.). The label may also be a phosphorescent compounds including porphyrins, phthalocyanines, polyaromatic compounds such as pyrenes, anthracenes and acenaphthenes, and so forth, may also be used.

Purification/Separation Tags

Examples include polyarginine, polyhistidine, or HATTM (Clontech), which is a naturally-occurring sequence of non-adjacent histidine residues that possess a high affinity for immobilized metal ions. Biopolymers comprising these polypeptides can be purified by, for example, affinity chromatography using immobilized nickel, zinc or TALON™ resin (Clontech), which comprises immobilized cobalt ions. See e.g. Knol et al. 1996 J Biol Chem 27(26) 15358-15366. Polypeptides comprising polyarginine allow effective purification by ion exchange chromatography. Other useful polypeptides include, for example, the antigenic identification peptides described in U.S. Pat. No. 5,011,912 and in Hopp et al. 1988 Bio/Technology 6 1204. One such peptide is the FLAG™ peptide, which is highly antigenic and provides an epitope reversibly bound by a specific monoclonal antibody, enabling rapid assay and facile purification of expressed recombinant fusion protein. A murine hybridoma designated 4E11 produces a monoclonal antibody that binds the FLAG peptide in the presence of certain divalent metal cations, as described in U.S. Pat. No. 5,011,912. The 4E11 hybridoma cell line has been deposited with the American Type Culture Collection under Accession No. HB 9259. Monoclonal antibodies that bind the FLAG peptide can be used as affinity reagents to recover a polypeptide purification reagent that comprises the FLAG peptide. Other suitable protein tags and affinity reagents are: 1) those described in GST-Bind™ system (Novagen), which utilizes the affinity of glutathione-S-transferase fusion proteins for immobilized glutathione; 2) those described in the T7-TAG* affinity purification kit (Novagen), which utilizes the affinity of the amino terminal 11 amino acids of the T7 gene 10 protein for a monoclonal antibody; or 3) those described in the STREP-TAG* system (Novagen), which utilizes the affinity of an engineered form of streptavidin for a protein tag. Some of the above-mentioned protein tags, as well as others, are described in Sassenfeld 1990 TIBTECH 8: 88-93, Brewer et al., in Purification and Analysis of Recombinant Proteins, pp.239-266, Seetharam and Sharma (eds.), Marcel Dekker, Inc. (1991), and Brewer and Sassenfeld, in Protein Purification Applications, pp. 91 - 111 , Harris and Angal (eds.), Press, Inc., Oxford England (1990). The portions of these references that describe protein tags are incorporated herein by reference. Further, fusions of two or more of the tags described above, such as, for example, a fusion of a FLAG tag and a polyhistidine tag, can be fused to a biopolymer of interest according to this disclosure.

Coupling/Purification Tags

A wide variety of appropriate coupling methods may be used to attach a biopolymers to a solid support. The coupling may be performed with covalent linkages such as amide linkages (e.g., amino NHS-ester), ester bonds, phosphoester bonds, or disulfide bonds. The coupling may also be performed using methods such as affinity tags, such as antigenic tags or other binding methods (e.g., antibody-protein A; biotin-streptavidin; FLAG-tag (Sigma-Aldrich, Hopp et al. 1988 Nat Biotech 6:1204-1210); glutathione S-transferase (GST)/glutathione; hemagluttanin (HA) (Wilson et al., 1984 Cell 37:767); intein fusion expression systems (New England Biolabs, USA) Chong et al. 1997 Gene 192(2), 271-281; maltose-binding protein (MBP)); poly His-(Ni or Co) (Gentz et al., 1989 PNAS USA 86:821-824); or thiol-gold. Fusion proteins containing GST-tags at the N-terminus of the protein are also described in U.S. Pat. No. 5,654,176 (Smith). Magnetic separation techniques may also be used such as Strepavidin-DynaBeads® (Life Technologies, USA). Alternatively, photo-cleavable linkers may be used, e.g., U.S. Pat. No. 7,595,198 (Olejnik & Rothchild). A wide variety of coupling methods, including polystyrene affinity peptides, are reviewed by Nakanishi et al. Nakanishi et al. 2008 Curr Proteomics 5 161-175, the contents of which and the other references of this section are hereby incorporated by reference in their entireties. Many other systems are known in the art and are suitable for use with the present disclosure.

Statistical Methods

The data may be ranked for its ability to distinguish biomarkers in both the 1 versus all (i.e., disease versus normal) and the all-pairwise (i.e., normal versus specific disease) cases. One statistic used for the ranking is the area under the receiver operator characteristic (ROC) curve (a plot of sensitivity versus (1-specificity)). Although biomarkers are evaluated for reliability across datasets, the independent sample sets are not combined for the purposes of the ROC ranking. As a result, multiple independent analyses are performed and multiple independent rankings are obtained for each biomarker's ability to distinguish groups of interest.

It is to be understood that other genes and/or diagnostic criteria may be used in this disclosure. For example, patient characteristics, standard blood workups, the results of imaging tests, and/or histological evaluation may optionally be combined with biomarkers disclosed herein.

Such analysis methods may be used to form a predictive model, and then use that model to classify test data. For example, one convenient and particularly effective method of classification employs multivariate statistical analysis modeling, first to form a model (a “predictive mathematical model”) using data (“modeling data”) from samples of known class (e.g., from subjects known to have, or not have, a particular class, subclass or grade of lung cancer), and second to classify an unknown sample (e.g., “test data”), according to lung cancer status.

Pattern recognition (PR) methods have been used widely to characterize many different types of problems ranging for example over linguistics, fingerprinting, chemistry and psychology. In the context of the methods described herein, pattern recognition is the use of multivariate statistics, both parametric and non-parametric, to analyze spectroscopic data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. The other approach is termed “supervised” whereby a training set of samples with known class or outcome is used to produce a mathematical model and is then evaluated with independent validation data sets.

Unsupervised PR methods are used to analyze data without reference to any other independent knowledge. Examples of unsupervised pattern recognition methods include principal component analysis (PCA), hierarchical cluster analysis (HCA), and non-linear mapping (NLM).

Alternatively, and in order to develop automatic classification methods, it has proved efficient to use a “supervised” approach to data analysis. Here, a “training set” of biomarker expression data is used to construct a statistical model that predicts correctly the “class” of each sample. This training set is then tested with independent data (referred to as a test or validation set) to determine the robustness of the computer-based model. These models are sometimes termed “expert systems,” but may be based on a range of different mathematical procedures. Supervised methods can use a data set with reduced dimensionality (for example, the first few principal components), but typically use unreduced data, with all dimensionality. In all cases the methods allow the quantitative description of the multivariate boundaries that characterize and separate each class, for example, each class of lung cancer in terms of its biomarker expression profile. It is also possible to obtain confidence limits on any predictions, for example, a level of probability to be placed on the goodness of fit (see, for example, Sharaf; Illman; Kowalski, eds. (1986). Chemometrics. New York: Wiley). The robustness of the predictive models can also be checked using cross-validation, by leaving out selected samples from the analysis.

Examples of supervised pattern recognition methods include the following nearest centroid methods (Dabney 2005 Bioinformatics 21(22):4148-4154 and Tibshirani et al. 2002 Proc. Natl. Acad. Sci. USA 99(10):6576-6572); soft independent modeling of class analysis (SIMCA) (see, for example, Wold, (1977) Chemometrics: theory and application 52: 243-282.); partial least squares analysis (PLS) (see, for example, Wold (1966) Multivariate analysis 1: 391-420; Joreskog (1982) Causality, structure, prediction 1: 263-270); linear discriminant analysis (LDA) (see, for example, Nillson (1965). Learning machines. New York.); K-nearest neighbor analysis (KNN) (see, for example, Brown and Martin 1996 J Chem Info Computer Sci 36(3):572-584); artificial neural networks (ANN) (see, for example, Wasserman (1993). Advanced methods in neural computing. John Wiley & Sons, Inc; O'Hare & Jennings (Eds.). (1996). Foundations of distributed artificial intelligence (Vol. 9). Wiley); probabilistic neural networks (PNNs) (see, for example, Bishop & Nasrabadi (2006). Pattern recognition and machine learning (Vol. 1, p. 740). New York: Springer; Specht, (1990). Probabilistic neural networks. Neural networks, 3(1), 109-118); rule induction (RI) (see, for example, Quinlan (1986) Machine learning, 1(1), 81-106); and, Bayesian methods (see, for example, Bretthorst (1990). An introduction to parameter estimation using Bayesian probability theory. In Maximum entropy and Bayesian methods (pp. 53-79). Springer Netherlands; Bretthorst, G. L. (1988). Bayesian spectrum analysis and parameter estimation (Vol. 48). New York: Springer-Verlag); unsupervised hierarchical clustering (see for example Herrero 2001 Bioinformatics 17(2) 126-136). In one embodiment, the classifier is the centroid based method described in Mullins et al. 2007 Clin Chem 53(7):1273-9, which is herein incorporated by reference in its entirety for its teachings regarding disease classification.

It is often useful to pre-process data, for example, by addressing missing data, translation, scaling, weighting, etc. Multivariate projection methods, such as principal component analysis (PCA) and partial least squares analysis (PLS), are so-called scaling sensitive methods. By using prior knowledge and experience about the type of data studied, the quality of the data prior to multivariate modeling can be enhanced by scaling and/or weighting. Adequate scaling and/or weighting can reveal important and interesting variation hidden within the data, and therefore make subsequent multivariate modeling more efficient. Scaling and weighting may be used to place the data in the correct metric, based on knowledge and experience of the studied system, and therefore reveal patterns already inherently present in the data.

If possible, missing data, for example gaps in column values, should be avoided. However, if necessary, such missing data may replaced or “filled” with, for example, the mean value of a column (“mean fill”); a random value (“random fill”); or a value based on a principal component analysis (“principal component fill”). Each of these different approaches will have a different effect on subsequent PR analysis.

“Translation” of the descriptor coordinate axes can be useful. Examples of such translation include normalization and mean centering. “Normalization” may be used to remove sample-to-sample variation. Many normalization approaches are possible, and they can often be applied at any of several points in the analysis. “Mean centering” may be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centred and unit variance scaled. This technique is a very useful because each descriptor is then weighted equally and large and small values are treated with equal emphasis. This can be important for analytes present at very low, but still detectable, levels.

Several supervised methods of scaling data are also known. Some of these can provide a measure of the ability of a parameter (e.g., a descriptor) to discriminate between classes, and can be used to improve classification by stretching a separation. For example, in “variance weighting,” the variance weight of a single parameter (e.g., a descriptor) is calculated as the ratio of the inter-class variances to the sum of the intra-class variances. A large value means that this variable is discriminating between the classes. For example, if the samples are known to fall into two classes (e.g., a training set), it is possible to examine the mean and variance of each descriptor. If a descriptor has very different mean values and a small variance, then it will be good at separating the classes. “Feature weighting” is a more general description of variance weighting, where not only the mean and standard deviation of each descriptor is calculated, but other well-known weighting factors, such as the Fisher weight, are used.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods and/or record the results may also be provided over an electronic network, for example, over the internet, an intranet, or other network.

The process of comparing a measured value and a reference value can be carried out in any convenient manner appropriate to the type of measured value and reference value for the discriminative gene at issue. “Measuring” can be performed using quantitative or qualitative measurement techniques, and the mode of comparing a measured value and a reference value can vary depending on the measurement technology employed. For example, when a qualitative colorimetric assay is used to measure expression levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the disclosure will most commonly be quantitative values. In other examples, measured values are qualitative. As with qualitative measurements, the comparison can be made by inspecting the numerical data, or by inspecting representations of the data (e.g., inspecting graphical representations such as bar or line graphs).

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a biomarker protein. Alternately, a separate device (e.g., a digital computer) may be used to compare the measured value(s) and the reference value(s). Automated devices for comparison may include stored reference values for the biomarker protein(s) being measured, or they may compare the measured value(s) with reference values that are derived from contemporaneously measured reference samples (e.g., samples from control subjects).

As will be apparent to those of skill in the art, when replicate measurements are taken, the measured value that is compared with the reference value is a value that takes into account the replicate measurements. The replicate measurements may be taken into account by using either the mean or median of the measured values as the “measured value.”

This disclosure also includes methods of identifying patients for particular treatments or selecting patients for which a particular treatment would be desirable or contraindicated.

The methods above may be performed by a reference laboratory, a hospital pathology laboratory or a doctor. The methods above may further comprise an algorithm and/or statistical analysis.

Unless defined otherwise, all technical and scientific terms used herein have the same meaning as commonly understood by one of ordinary skill in the art to which this disclosure belongs. The article “a” and “an” are used herein to refer to one or more than one (i.e., to at least one) of the grammatical object(s) of the article. By way of example, “an element” means one or more elements.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

The following examples further illustrate the disclosure and are not intended to limit the scope of the disclosure. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### EXAMPLES

Detailed Description of the Drawings

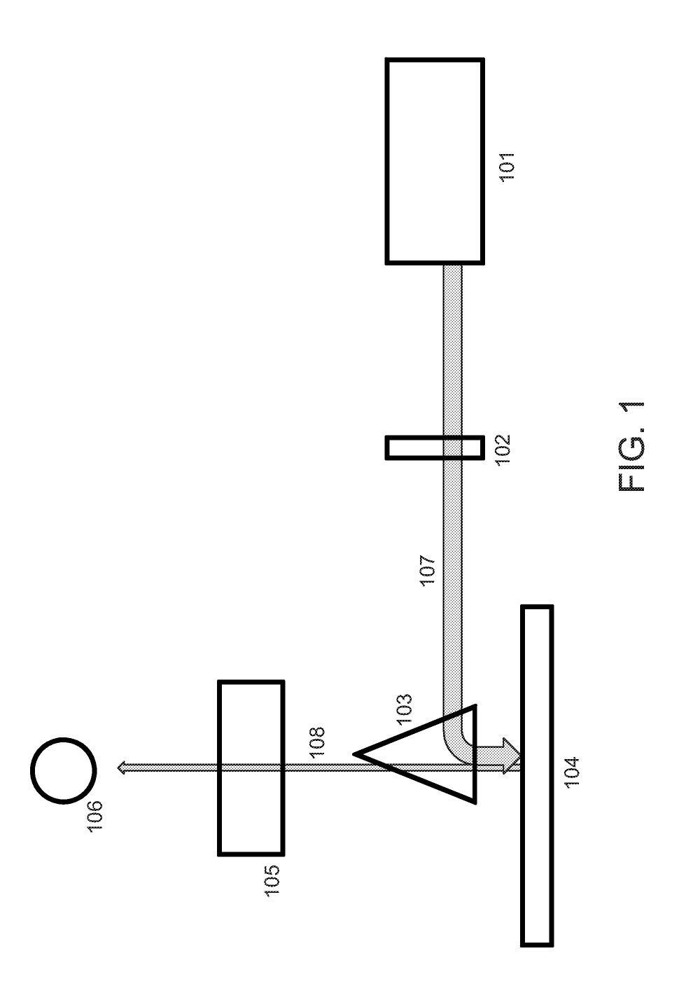

FIG. 1—Excitation light (107) is produced by the light source (101). It then passes through the excitation filter (102). The purpose and/or existence of this filter will vary based upon the exact light source used but the goal is to ensure that a narrowly defined, user specified wavelength is used to excite the labeled nucleic acid. Once the defined light passes through the filter it encounters a dichroic mirror (103). The purpose of this mirror is to direct the excitation light downward toward the sample (104) preferably containing nucleic acids such as DNA. In its most common iteration the DNA will be in a commercially available platform such as a 96, 384 or 1536 well plate. Other platforms include beads, fibers, and assorted solid or semi-solid supports. After the excitation light reaches the nucleic acid with fluorescent moieties incorporated, light is emitted (108). The emitted light then reaches the tunable filter (105). This tunable filter could be an acoustic tunable filter, a filter wheel, a diffraction gradient or liquid crystal filters. All of these technologies have different tuning methods. For an example of a filter wheel, see U.S. Pat. No. 8,496,879 (Atzler), the contents of which is hereby incorporated in its entirety. Preferably, the tunable filter is a liquid crystal filter. However regardless of the method used to tune the filter, all of these technologies are capable of delivering specified wavelengths to the detector (106). The detector can be either a photomultiplier tube (PMT) or a charge coupled device CCD. The sequential arrival of specific predetermined wavelengths to the detector can be used to create a spectral profile for each geography. These spectral profiles can then be used to determine which fluorescent moieties are present at a specific geography even in cases where their fluorescence or the auto-fluorescence of the materials is the predominate signal reaching the detector.

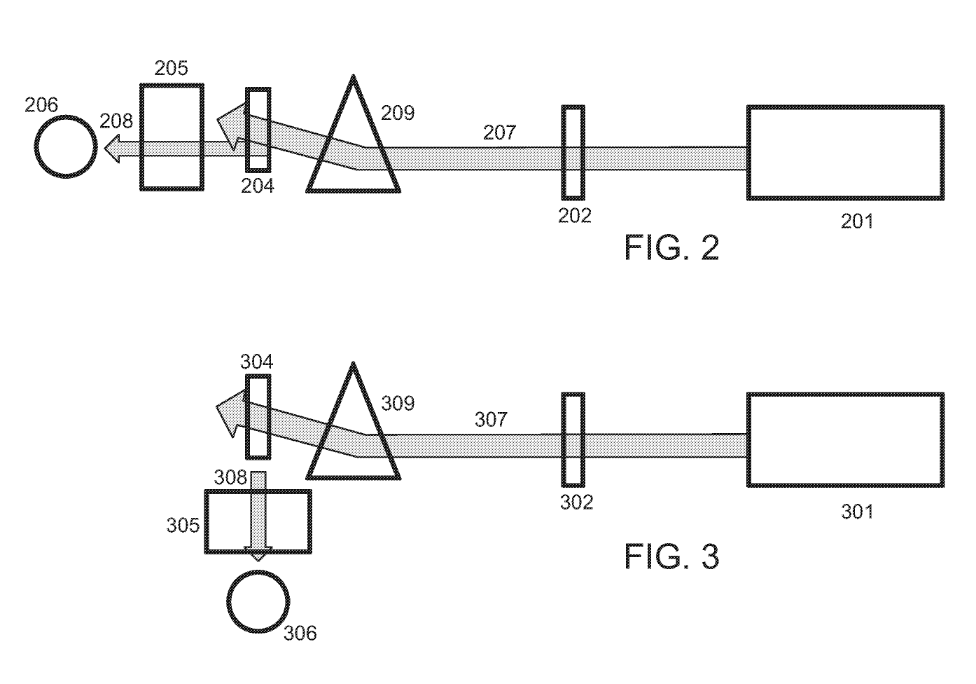

FIG. 2 shows an alternative embodiment. Here, excitation light (207) is produced by the light source (201). It then passes through the excitation filter (202). Once the defined light passes through the filter in one embodiment of the technology the light will encounter a multi-part prism (209). Here, the prism has two functions: first the prism is designed to alter the light path so that the excitation light does not reach the detector which is positioned 180 relative to the emission light source. Second this prism will be movable along the “Z” axis. As such by moving the prism the light path will be directed along the “Z” axis of the DNA. In other iterations of the technology the light will encounter any of number of different materials which under mechanical force or electrical current will bend the path of the light. These materials will serve the same two functions of directing the excitation light away for the detector and along the “Z” axis of the DNA. After the excitation light reaches the sample (204), e.g., nucleic acid with fluorescent moieties incorporated, light is emitted (208). This light then reaches the tunable filter (205). Next, the emitted light from the sample passes through the tunable filter (205) and specified wavelengths are delivered to the detector (206).

FIG. 3 shows another alternative embodiment. In this embodiment, excitation light (307) is produced by the light source (301). It then passes through the excitation filter (302). The purpose and/or existence of this filter will vary based upon the exact light source used but the goal is to ensure that a narrowly defined, user specified wavelength is used to excite the labeled nucleic acid. Once the defined light passes through the filter in one iteration of the technology the light will encounter a movable multi-part prism (309). The purpose of this prism is to provide the ability to assay the sample along the “Z” axis that is perpendicular to the X and Y axis of a plate containing multiple wells. As such by moving the prism the light path will be directed along the “Z” axis of the sample containing the nucleic acids. In other iterations of the technology the light will encounter any one of number of different materials which under mechanical force or electrical current will bend the path of the light. These materials will serve the same two functions of directing the excitation light away for the detector and along the “Z” axis relative to a plate containing multiple samples each with a different combination of labels on the nucleic acid to be analyzed. After the excitation light reaches the DNA with fluorescent moieties incorporated, light is emitted (308). This light then reaches the tunable filter (305) positioned approximately perpendicular to the light source (307). Next, the emitted light from the sample passes through the tunable filter (305) and specified wavelengths are delivered to the detector (306).

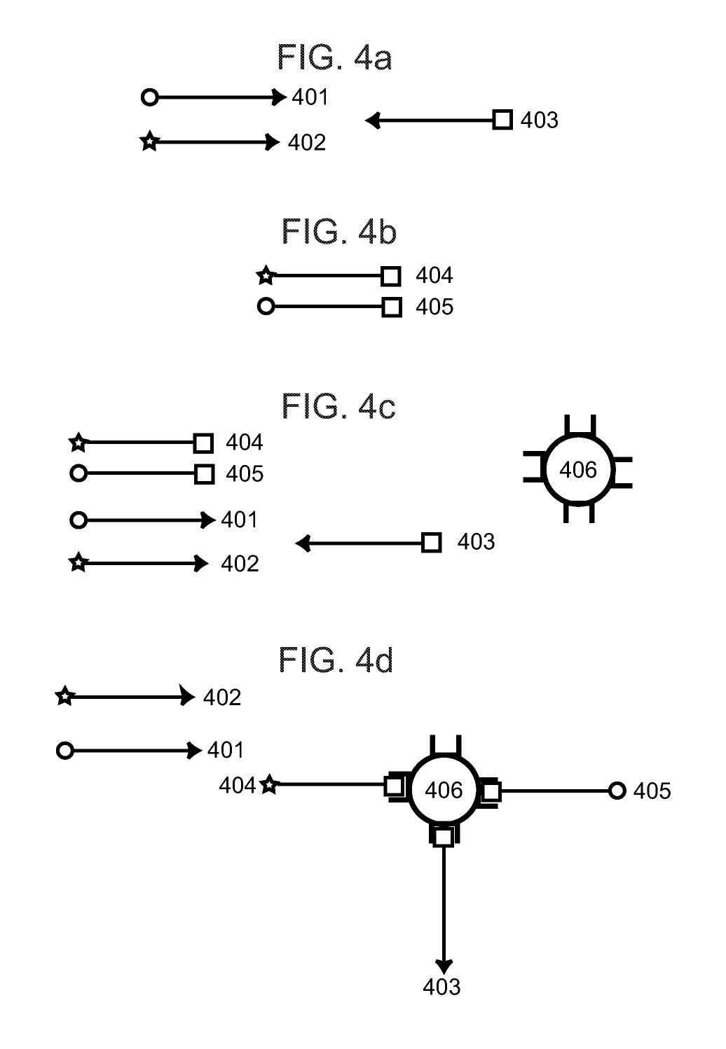

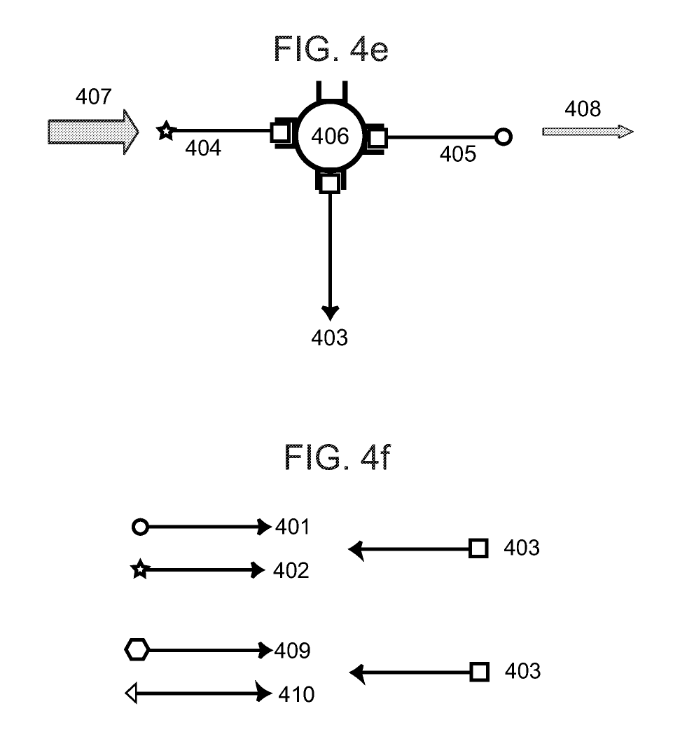

FIG. 4a-4f—Throughout this description primers will be referred to by their functional moieties without regard to specific sequences. If primers contain the same functional groups, it will receive the same numbering even if the underlining sequences are different. As seen in FIG. 4a, in one embodiment of the technology there are three primers: two forward primers (401) and (402) and one reverse primer (403). The forward primers have three parts: on the 5′ most part of the primer there is a fluorescent moiety, in the middle there is a sequence which specifies the genomic region of interest and on the 3′ most end there is a sequence to specify which of the two possible SNPs/molecular markers corresponds to the primer. For one primer (401) there is one fluorescent moiety represented by a circle; for the other primer (402) there is a different fluorescent moiety represented by a star. In one embodiment, in the 3′ end of each forward primer there is a different nucleotide designed to detect a single nucleotide polymorphism (SNP). Thus the 3′ end of the forward primer is used to differentiate between the two SNPs present on the genomic DNA while the 5′ end of the forward primer will be labeled with a specific fluorophore to chosen to identify a particular SNP. For a PCR reaction to function there also has to be a reverse primer (403). In our case the reverse primer will have a functional group (represented as a square) on its' 5′ end. As seen in FIG. 4b, there are two possible amplicons that may be generated in the PCR reaction (404) and (405). However as seen in FIG. 4c there are also unincorporated primers present in the PCR reaction. These unincorporated primers will provide unacceptable levels of background. In order to eliminate this background we will select for the amplicons on the basis of a tag present on the reverse primer. As seen in FIG. 4d, this tag or functional group will be used to purify the amplicons (404) and (405) away from the unincorporated fluorescent primers (401) and (402) after a PCR reaction is completed. For example in one embodiment of this technology the tag is a biotin (403) and for this embodiment, streptavidin beads (406) are be used to purify the amplicons (404) and (405) away from the unincorporated primers (401) and (402). This will create an amplicon decorated bead, see FIG. 4d. In FIG. 4e, these beads (406) with bound amplicons (404) and (405) will then be imaged in the apparatus described in FIGS. 1, 2 and 3. Excitation light (407) will strike the bead and emission light (408) will be produced. The geometry of the excitation and emission light beams will depend upon on the configurations disclosed in FIG. 1, 2 or 3. FIGS. 4a-4e represent the steps required to identify and discriminate a single SNPs. FIG. 4f describes a method to identify multiple SNPs, within a single PCR reaction and under a single selection principle. This functionality is provided by adding additional fluorescent moieties to the 5′ end of the oligo. Specifically, adding an additional pair of forward primers with sequences for additional SNPs (409) and (410) to the PCR reaction provides the needed means to identify and discriminate another different SNP by amplifying different sequences than those identified by forward primers (401) and (402) and discriminating based upon different fluorescent moieties.

To one of ordinary skill, the advantages of the apparatus described herein become apparent. Unlike traditional optical configurations, the disclosed apparatus and method allows one to use fluorophores with overlapping spectra. Theoretically it is possible to utilize and identify hundreds of different fluorophores in the same geography even if all the fluorophores had the exact same peak emission. Therefore it is possible to discriminate hundreds of different SNPs/molecular markers using the same selection criteria.

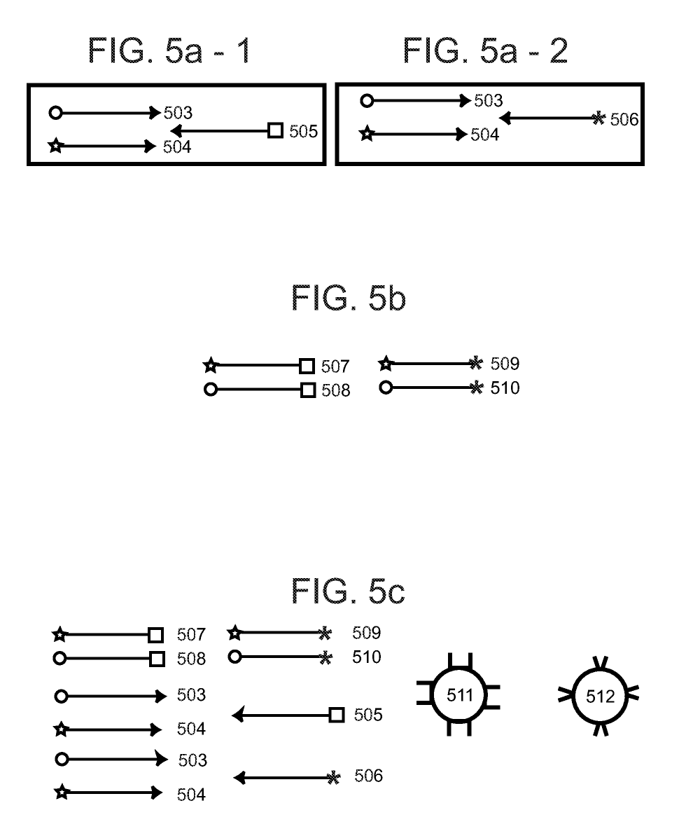

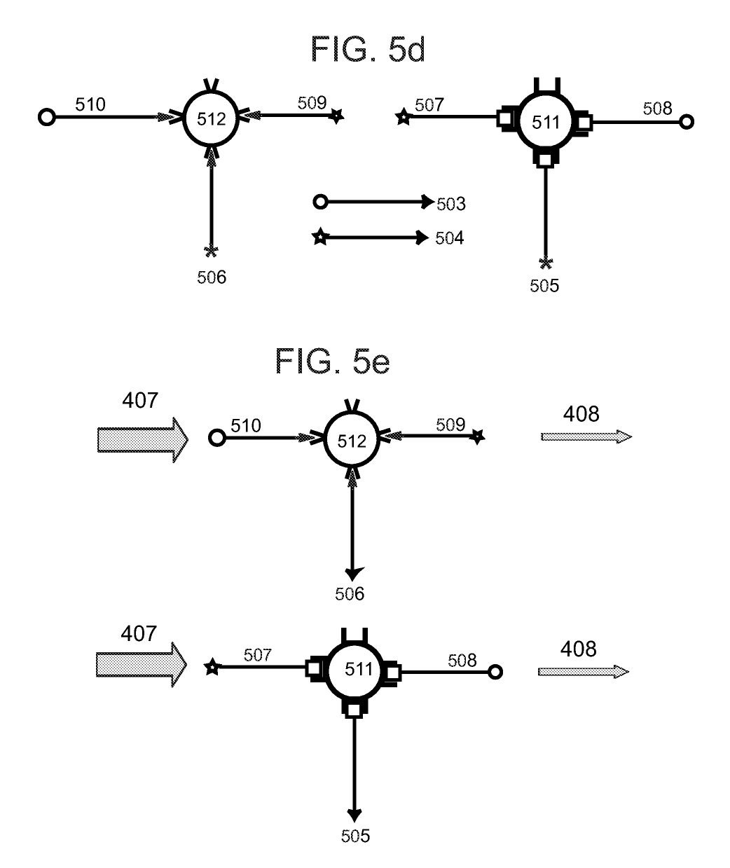

FIG. 5a-5e. Throughout this description primers will be referred to by their functional moieties without regard to specific sequences. If the primers contain the same functional groups, they will receive the same numbering even if the underlining sequences are different. As seen in FIG. 5a, in one embodiment of the technology there are multiple groupings of three primers. In FIG. 5a-1, there are two forward primers (503) and (504) and one reverse primer (505). In FIG. 5a-2, there are two forward primers (503) and (504) and a different reverse primer (506). In both FIG. 5a-1 and FIG. 5a-2, the forward primers have three parts: on the 5′ most part of the primer there is a fluorescent moiety, in the middle there is a sequence which specifies the genomic region of interest and on the 3′ most end there is a sequence to specify which of the two possible SNPs/molecular markers corresponds to the primer. For one primer (503) there is one fluorescent moiety represented by a circle; for the other primer (504) there is a different fluorescent moiety represented by a star. On the 3′ end of each forward primer there is a different nucleotide depending upon the SNP/molecular marker. Thus the 3′ end of the primer is used to differentiate between the two SNPs present in the genomic DNA while the 5′ end of the primer is used to assign a specific fluorophore to that SNP. For a PCR reaction to function there also has to be a reverse primer (505). In our case the reverse primer will have a functional group on its' 5′ end. The functional group on the reverse primer will be specific to the grouping. So primer (505) in FIG. 5a-1 will have one functional purification element while primer (506) in FIG. 5a-2 will have different functional purification element. It is important to note that although in this illustration within each grouping there are the minimal requirements for the identification of a single SNP/molecular marker, it is possible to identify multiple SNPs within a grouping by adding additional fluorescent moieties as disclosed in FIG. 4f.

In FIG. 5b, the different amplicons that will be produced by the PCR reaction can be seen (507), (508), (509), and (510). Amplicons (507) and (508) have the same purification element relative to each other but different fluorophores relative to each other. In other words (507) and (508) will localize to the same geography but have different colors. Amplicons (509) and (510) have the same purification element relative to each other but different fluorophores relative to each other. Amplicons (509) and (510) will localize to same geography but have different colors than each other. Amplicons (507) and (509) have the same fluorophores relative to each other but different purification elements. In other words amplicons (507) and (509) have the same colors but different geographies. Similarly amplicons (508) and (510) have the same fluorophores but different geographies. By selecting different purification elements and fluorophores it is possible to provide a unique combination of both geography and color for each SNP in a PCR reaction.

As shown in FIG. 5c there are also unincorporated primers present in the PCR reaction. These unincorporated primers will provide unacceptable levels of background. In order to eliminate this background we will select for the amplicons on the basis of the functional group present on the reverse primer. As seen in FIG. 5d, the functional groups will be used to purify the amplicons (507), (508), (509), and (510) away from the unincorporated fluorescent primers (503) and (504) after a PCR reaction is completed. For example in one embodiment of this technology the functional group on the amplicons (507) and (508) will be biotin. For this embodiment, streptavidin beads (511) will be used to purify the amplicons (507) and (508) away from the unincorporated fluorescent primers (503) and (504) (Green, N. M. (1975) Adv. Protein Chem. 29, 85-143). Continuing with this example, the other functional group present on the amplicons (509) and (510) could be a peptide. An antibody decorated bead (512) could be used to isolate the amplicons (509), and (510) from the fluorescent primers (503) and (504) (Pauling, Linus, et al (1943) Science 98, 263-264). As seen in FIG. 5d this will create beads (511) and (512) bound to differing sets of amplicons. As seen in FIG. 5e, these beads (511) and (512) will then be imaged in the MSI apparatus seen in FIGS. 1, 2, and 3. There are several different ways to differentiate the beads between each other. The autofluorescence of the beads could be used to differentiate the beads. The beads could be colored during their manufacturing process. Specific combinations of fluorophores/purification elements could be chosen that enable discrimination between the beads. A positive control consisting of a dual labeled fluorophore/purification element could be added to the mastermix. This is an especially attractive way of discriminating between the beads since it will provide additional information about the purification processes that were employed. Finally the beads themselves could be replaced by a support better suited to providing geographic information. This support could be a fiber, a microtiter plate with spotted elements on the base of it, a slide, or a polymer coated metallic element.

It is to be understood that, while this disclosure has been described in conjunction with the detailed description, thereof, the foregoing description is intended to illustrate and not limit the scope of the claims. Other aspects, advantages, and modifications of this disclosure are within the scope of the claims set forth below. All publications, patents, and patent applications cited in this specification are herein incorporated by reference as if each individual publication or patent application were specifically and individually indicated to be incorporated by reference.

What is claimed is:

1. A system for analyzing multiple labeled nucleic acids in a sample, each labeled nucleic acids being labeled by a covalent linkage with at least one synthetic fluorescent molecule capable of generating emitted light at one or more light frequencies in response to an exciting light, the system comprising: a light source configured to produce an exciting light; an excitation filter; a dichroic mirror capable of directing the exciting light to the sample; a tunable emitted light filter capable of filtering the plurality of emitted light frequencies from the sample; and a detector capable of detecting the plurality of emitted light frequencies from the multiple labeled nucleic acids in the sample wherein the tunable emitted light filter is at about a 0 to 45 degree angle or a 135 to 180 degree angle to the directed exciting light and the sample.

2. The system of claim 1, wherein the tunable emitted light filter is about a 5 to 25 degree angle to the directed exciting light and the sample.

3. The system of claim 2, wherein the tunable emitted light filter is about a 10 to 20 degree angle to the directed exciting light and the sample.

4. The system of claim 1, wherein the tunable emitted light filter is about a 155 to 175 degree angle to the directed exciting light and the sample.

5. The system of claim 1, wherein the tunable emitted light filter is about a 160 to 170 degree angle to the directed exciting light and the sample.

6. The system of claim 1, wherein the tunable emitted light filter is a solid state tunable light filter.

7. The system of claim 6, where the solid state tunable light filter is a liquid crystal tunable light filter.

8. The system of claim 1, wherein each labeled nucleic acid further comprises a purification tag.

9. The system of claim 8, wherein the purification tag is a synthetic polypeptide designed to bind a specific antibody or a metal binding polypeptide.

10. The system of claim 1, wherein the light source is a plurality of lasers.

11. A system for analyzing multiple labeled nucleic acids in a sample, the labeled nucleic acids being capable of generating emitted light at a plurality of light frequencies in response to an exciting light, the system comprising: a light source configured to produce an exciting light; an excitation filter; a multi-part prism capable of directing the exciting light to the sample; a tunable emitted light filter capable of filtering the plurality of emitted light frequencies from the sample; and a detector capable of detecting the plurality of emitted light frequencies from the multiple labeled nucleic acids in the sample.

12. A method of analyzing multiple labeled nucleic acids in a sample, the labeled nucleic acids being capable of generating a plurality of emitted light frequencies in response to an exciting light, the method comprising: producing an exciting light from a light source; directing the light source through an excitation filter; directing the filtered excitation light through a dichroic mirror capable of directing the exciting light to the sample; generating the plurality of emitted light frequencies from the sample and directing the emitted light to a tunable emitted light filter; directing the filtered emitted light to a detector capable of detecting plurality of emitted light frequencies from the multiple labeled nucleic acids in the sample.

13. The method of claim 12, wherein the multiple labeled nucleic acids are multiple labeled DNA molecules.

14. The method of claim 13, wherein the multiple labeled DNA molecules are amplicons from a PCR reaction.

15. The method of claim 12, wherein the multiple labeled nucleic acids are designed to be specific for a single nucleotide polymorphism (SNP).

16. The method of claim 12, wherein the sample is a bead or solid substrate.

17. The method of claim 12, wherein the bead has a plurality of nucleic acids non-covalently bound to the bead or solid substrate.

18. The method of claim 12, wherein the sample is a homogenous solution in a multi-welled plate.

19. The method of claim 12, wherein the sample is a homogenous solution in a multi-welled sampling device.
